(19) United States
(12) Patent Application Publication
(10) Pub. No.: US 2015/0283020 A1
(43) Pub. Date: Oct. 8, 2015

(54) WALKING INTENTION DETECTION DEVICE AND SYSTEM AND METHOD THEREOF

(71) Applicant: Hyundai Motor Company, Seoul (KR)

(72) Inventors: Hyung Joo Kim, Busan (KR); Dong Jin Lee, Seoul (KR)

(21) Appl. No.: 14/462,947

(22) Filed: Aug. 19, 2014

(30) Foreign Application Priority Data
Apr. 4, 2014 (KR) 10-2014-0040443

(51) Int. Cl. A61H 3/00; A61F 2/68; A61F 2/60; A61B 5/11

(52) U.S. Cl. CPC A61H 3/00; A61B 5/11; A61F 2/68; A61F 2/60; A61F 2002/607; A61F 2002/608

(57) ABSTRACT

A walking intention detection device and a system and a method thereof are provided. The walking intention detection device includes a lower wearing part into which a walker's foot is inserted and an upper wearing part disposed over the lower wearing part to cover a top of a foot and a heel of a walker. A tension sensor is disposed between the lower wearing part and the upper wearing part and is configured to measure a force delivered to the lower wearing part and the upper wearing part in response to a walking motion. In addition, a walking intention is detected when the wearable robot is used to provide the stability of the walking operation, thereby improving the marketability and convenience and the walking performance of the wearable robot is increased by the simplified mechanism, thereby creating the higher efficiency at the low costs.

### CROSS-REFERENCE TO RELATED APPLICATION

This application is based on and claims the benefit of priority to Korean Patent Application No. 10-2014-0040443, filed on Apr. 4, 2014 in the Korean Intellectual Property Office, the disclosure of which is incorporated herein in its entirety by reference.

### TECHNICAL FIELD

The present disclosure relates to a walking intention detection device and a system and a method thereof, and more particularly, to a walking intention detection device and a system and a method thereof that detect a walking intention of a walker mounted with a wearable robot.

### BACKGROUND

Generally, a human body wearable walking robot is a wearable robot using human-robot synchronization. Recently, research to detect walking intention for a walking operation of a walking robot with the development of such a technology has been conducted. The walking intention is considered to be a human walking intention, and when the walking intention may not be directly confirmed, the walking intention may be inferred based on a change in a specific body part (e.g., a body movement). In addition, a walking intention detection means that a user's intention to start or stop walking is detected or means that a motion state of left and right feet is detected.

Meanwhile, the devices in the related art detect the walking intention based on what operation the user desires to perform in the human wearable walking assist robot using a sensor or a load cell mounted on a robot foothold or an electromyogram. However, in the case of using the sensor or the load cell mounted on the foothold, durability or accuracy is reduced and when using the electromyogram, discrimination may be reduced at the time of a muscle fatigue and the accuracy for the walking intention may be reduced.

### SUMMARY

The present disclosure provides a walking intention detection device and a system and a method thereof that detect walking intention of a walker mounted with a wearable robot.

According to an exemplary embodiment of the present disclosure, a walking intention detection device of a walker may include: a lower wearing part into which a walker's foot is inserted; an upper wearing part configured to be provided over the lower wearing part to cover a top of a foot and a heel of a walker; and a tension sensor disposed between the lower wearing part and the upper wearing part and configured to measure a force delivered to the lower wearing part and the upper wearing part in response to a walking motion.

The walking intention detection device may further include: a controller configured to be linked with the tension sensor to detect the walking intention. The upper wearing part may include a first upper wearing part configured to cover the top of the foot and a second upper wearing part configured to cover the heel. The first upper wearing part and the second upper wearing part may be spaced apart from each other at a predetermined interval. The tension sensor may include: a first load cell disposed between front portions of the first upper wearing part and the lower wearing part; and a second load cell disposed between rear portions of the second upper wearing part and the lower wearing part.

Further, the first load cell may include a first connection band and the second load cell may include a second connection band, and thus the first connection band of the first load cell may connect the first upper wearing part with the lower wearing part and the second connection band of the second load cell may connect the second upper wearing part with the lower wearing part. The tension sensor may further include: a third load cell disposed between the rear portion of the first upper wearing part and the rear portion of the lower wearing part to connect the rear portion of the first upper wearing part with the rear portion of the lower wearing part via a third connection band; and a fourth load cell disposed between the front portions of the second upper wearing part and the lower wearing part to connect the front portion of the second upper wearing part with the front portion of the lower wearing part via a fourth connection band. The third load cell and the fourth load cell may be spaced apart from each other and the third connection band and the fourth connection band may intersect each other when spaced apart from each other.

Additionally, rectangular frames may be disposed between the front portions of the first upper wearing part and the lower wearing part and the rear portions of the second upper wearing part and the lower wearing part and an inside of the rectangular frame may include the third load cell, the third connection band, the fourth load cell, and the fourth connection band. The tension sensors may be disposed on an outer side and an inner side of the lower wearing part and the upper wearing part, respectively, to measure a balance of force generated from the foot during the walking motion.

According to another exemplary embodiment of the present disclosure, a walking intention detection system of a walker may include: an input unit configured to sense and measure generated information using a tension sensor in a wearing part into which a walker's foot is inserted; an amplifier configured to amplify the information generated from the input unit into a signal; a calculator configured to detect the walking intention with the signal amplified by the amplifier; and an output unit configured to operate a robot in response to the walking intention detected by the calculator.

According to another exemplary embodiment of the present disclosure, a walking intention detection method of a walker may include: sensing and measuring generated information using a tension sensor in a wearing part into which a walker's foot is inserted; and operating a robot based on the information detected in the sensing and measuring of the information. The walking intention detection method may further include: amplifying the information generated in the sensing and measuring of the information into a signal; and detecting a walking intention with the signal amplified in the amplifying of the information to operate the robot in response to the detected walking intention.

### BRIEF DESCRIPTION OF THE DRAWINGS

The above and other objects, features and advantages of the present disclosure will be more apparent from the following detailed description taken in conjunction with the accompanying drawings.

FIG. 1 is an exemplary diagram illustrating a walking intention detection device of a walker according to an exemplary embodiment of the present disclosure;

FIG. 2 is an exemplary side view illustrating the walking intention detection device of the walker according to the exemplary embodiment of the present disclosure;

FIGS. 3A-3B are exemplary diagrams illustrating a state before a force is generated in the walking intention detection device of the walker according to an exemplary embodiment of the present disclosure;

FIGS. 4A-4B are exemplary diagrams illustrating a state in which a force is generated during a walking motion in the walking intention detection device of the walker according to the exemplary embodiment of the present disclosure;

FIGS. 5A-5B are exemplary diagrams illustrating a state in which a force is generated during a reverse walking motion in the walking intention detection device of the walker according to the exemplary embodiment of the present disclosure;

FIG. 6 is an exemplary configuration diagram illustrating a walking intention detection system of a walker according to an exemplary embodiment of the present disclosure; and

FIG. 7 is an exemplary diagram illustrating a walking intention detection method of a walker according to an exemplary embodiment of the present disclosure.

### DETAILED DESCRIPTION

Although exemplary embodiment is described as using a plurality of units to perform the exemplary process, it is understood that the exemplary processes may also be performed by one or plurality of modules. Additionally, it is understood that the term controller refers to a hardware device that includes a memory and a processor. The memory is configured to store the modules and the processor is specifically configured to execute said modules to perform one or more processes which are described further below.

Furthermore, control logic of the present invention may be embodied as non-transitory computer readable media on a computer readable medium containing executable program instructions executed by a processor, controller or the like. Examples of the computer readable mediums include, but are not limited to, ROM, RAM, compact disc (CD)-ROMs, magnetic tapes, floppy disks, flash drives, smart cards and optical data storage devices. The computer readable recording medium can also be distributed in network coupled computer systems so that the computer readable media is stored and executed in a distributed fashion, e.g., by a telematics server or a Controller Area Network (CAN).

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

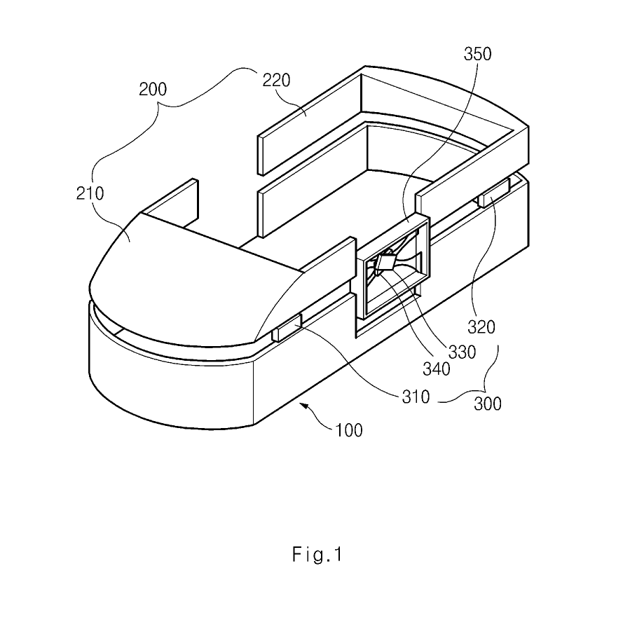

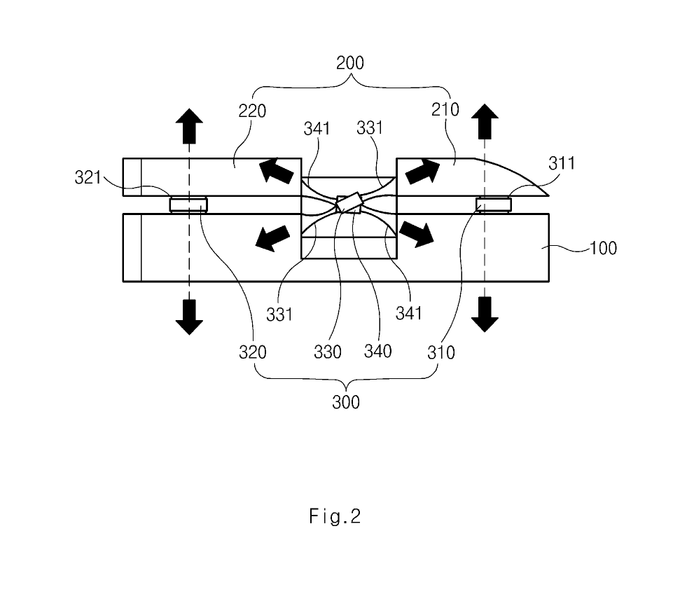

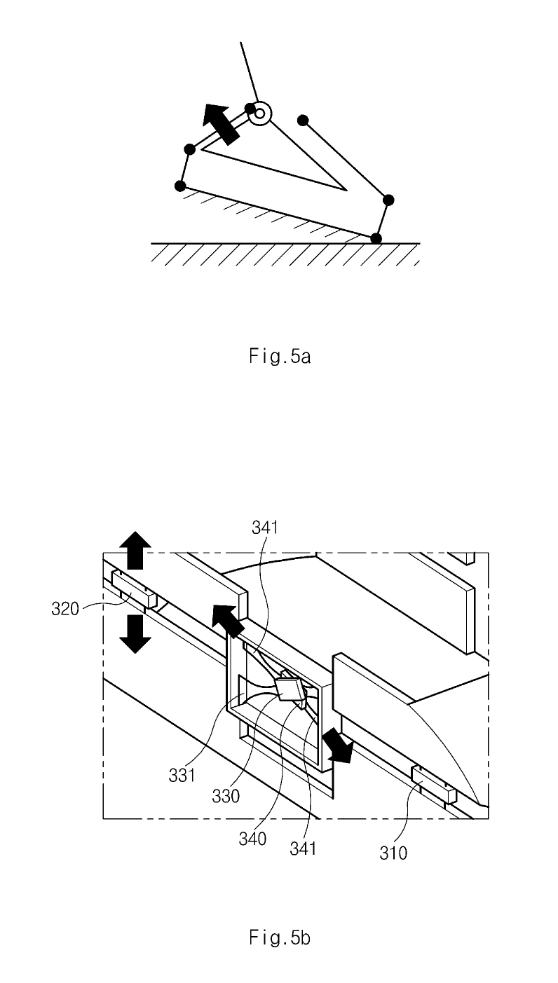

Exemplary embodiments of the present disclosure will be described in detail with reference to the accompanying drawings. As illustrated in FIGS. 1 to 5, a walking intention detection device of a walker according to an exemplary embodiment of the present disclosure may include a lower wearing part 100 configured to accommodate a walker's foot, an upper wearing part 200 over the lower wearing part 100, and a tension sensor 300 configured to measure a force delivered to the lower wearing part 100 and the upper wearing part 200 during a walking motion. As illustrated in FIGS. 1 and 2, the lower wearing part 100 may have a shoe shape of which the upper surface and may be opened to accommodate a walker's foot therein. In this configuration, the lower wearing part 100 may have a substantially flat bottom to allow a walker's sole of the foot to contact a bottom surface of the lower wearing part 100 and may include two sides, a rear surface, and front and rear portions which may include a side wall having a predetermined height.

Meanwhile, the lower wearing part 100 may be formed to be greater than a general shoe shape to allow the walker's foot to more easily enter into and exit therefrom. The upper wearing part 100 may be disposed over the lower wearing part 100 to cover a top of the foot or the heel of a walker. In particular, the upper wearing part 200 may include a first upper wearing part 210 configured to cover the walker's top of the foot and a second upper wearing part 220 configured to cover the walker's heel, in which the first upper wearing part 210 and the second upper wearing part 220 may be spaced apart from each other at a predetermined interval.

Further, the front portion of the first upper wearing part 210 may have a shape similar to the top of the foot to cover the walkers top of the foot, the rear portion of the second upper wearing part 220 may include a locked part to cover the walker's heel, and both sides of the first upper wearing part 210 and the second upper wearing part 220 may include a side wall having a predetermined height. The tension sensor 300 may be disposed between the lower wearing part 100 and the upper wearing part 200 to be configured to measure the force delivered to the lower wearing part 100 and the upper wearing part 200 based on the walking motion and to detect the walking intention.

The walking intention detection device of a walker may further include a controller connected to the tension sensor 300 and configured to detect the walking intention. In this configuration, the controller may be disposed at the upper wearing part 200 or the lower wearing part 100, but may be separately disposed at the outside of the device to be wirelessly linked with the tension sensor. In other words, according to the exemplary embodiment of the present disclosure, the walker's foot may be mounted in the wearing part corresponding to the robot's foot when the wearable robot is mounted to measure a force using the tension sensor 300 and the measured data may be transmitted to the controller to detect the walking intention, thereby operating the robot in response to the walking intention.

The tension sensor 300 may include a first load cell 310 disposed between the front portions of the first upper wearing part 210 and the lower wearing part 100 and a second load cell 320 disposed between the rear portion of the second upper wearing part 220 and the rear portion of the lower wearing part 100 to detect whether the force delivered from the walker's foot is generated from the top of the foot or the heel at the time of walking. Accordingly, the first load cell 310 may include a first connection band 311 and the second load cell 320 may include a second connection band 321, such that the first connection band 311 of the first load cell 310 connects the first upper wearing part 210 with the lower wearing part 100 and the second connection band 321 of the second load cell 320 connects the second upper wearing part 220 with the lower wearing part 100. In this configuration, when the force delivered from the walker's foot is the top of the foot at the time of walking, the first connection band 311 disposed between the first upper wearing part 210 and the lower wearing part 100 may be tensioned (e.g., tension may be detected on the first connection band 311) and the first load cell may be configured to detect the tensioned first connection band to detect that a force generating point is the top of the foot. Additionally, when the force delivered from the walker's foot is the heel, the second connection band 321 disposed between the second upper wearing part 220 and the lower wearing part 100 may be tensioned (e.g., taut) and the second load cell 320 may be configured to detect the tensioned second connection band to detect that a force generating point is the heel.

Further, the tension sensor part 300 may further include a third load cell 330 disposed between the rear portion of the first upper wearing part 210 and the rear portion of the lower wearing part 100 to connect the rear portion of the first upper wearing part 210 with the rear portion of the lower wearing part 100 via a third connection band 331 and a fourth load cell 340 disposed between the front portions of the second upper wearing part 220 and the lower wearing part 100 to connect the front portion of the second upper wearing part 220 with the front portion of the lower wearing part 100 via a fourth connection band 341. In particular, the third load cell 330 and the fourth load cell 340 may be spaced apart from each other and the third connection band 331 and the fourth connection band 341 may intersect each other while spaced apart to detect that when the walker moves forward or reversely (e.g., in a forward or in a reverse/backward direction), the top of the foot or the heel moves in a diagonal direction.

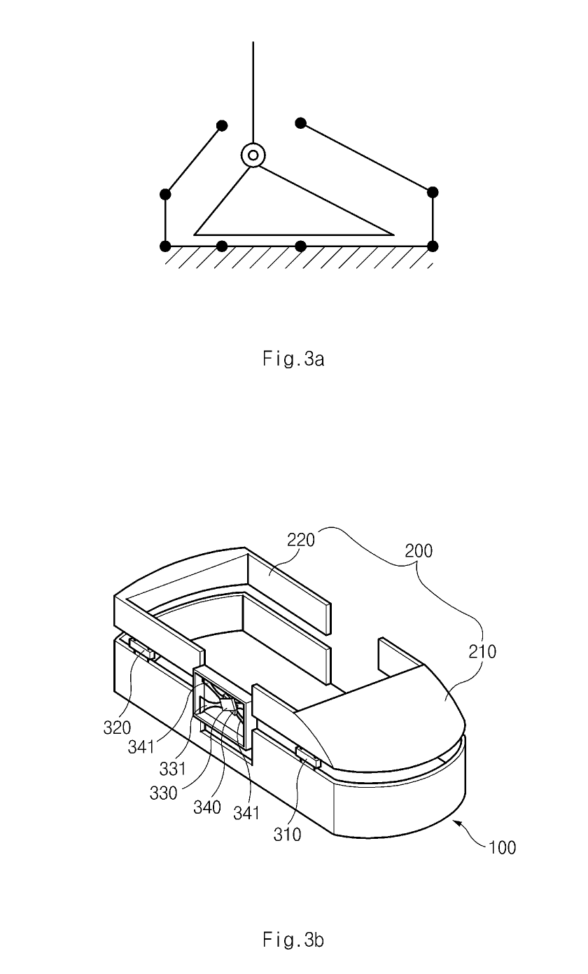

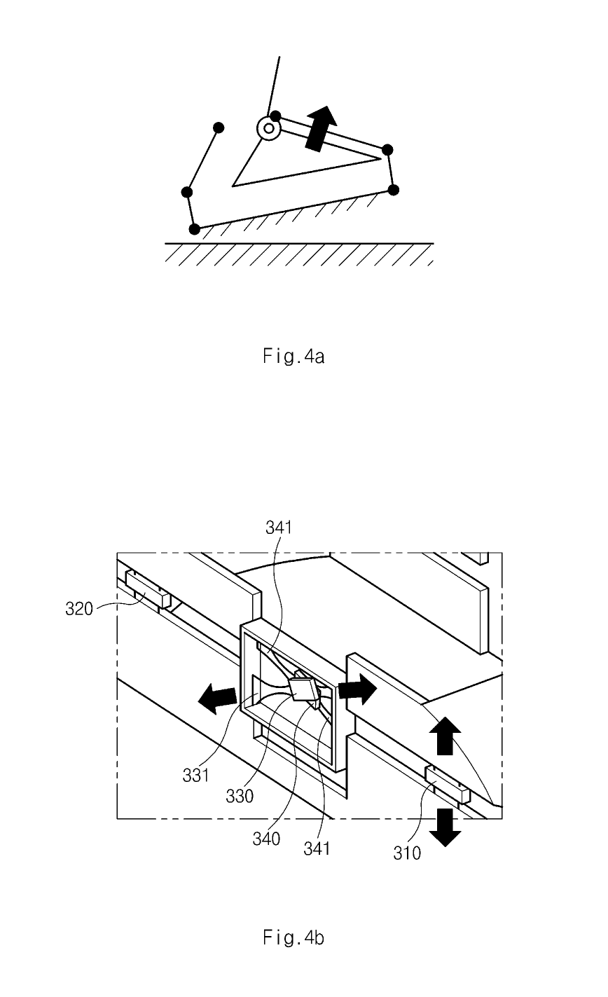

As described above, according to the exemplary embodiment of the present disclosure, the connection band disposed between the lower wearing part 100 and the upper wearing part 200 may be selectively tensioned due to the generation of the force between the lower wearing part 100 and the walker's foot and between the upper wearing part 200 and the walker's foot at the time of walking (e.g., during a walking motion) and as illustrated in FIGS. 3A to 4B, during a forward movement, the first connection band 311 mounted at the front surface of the first upper wearing part 210 may be vertically tensioned and the third connection band 331 of an upper end of a tip of the top of the foot and a lower end of the heel may be tensioned, due to a force to have the walker's top of the foot push the first upper wearing part 210. Further, as illustrated in FIGS. 3A to 5B, at the time of moving reversely (e.g., while walking in a reverse direction), the second connection band 321 mounted at the rear surface of the second upper wearing part 220 may be tensioned and the fourth connection band 341 of the upper end of the heel and a lower end of a tip of a toe of the second upper wearing part 220 may be selectively tensioned, due to a force to have the walker's heel push the second upper wearing part 220.

Moreover, rectangular frames 350 may be disposed between the front portions of the first upper wearing part 210 and the lower wearing part 100 and the rear portions of the second upper wearing part 220 and the lower wearing part 100 and thus the third load cell 330 and the third connection band 331 and the fourth load cell 340 and the fourth connection band 341 may be disposed inside the rectangular frame 350. In addition, the tension sensor 300 may be selectively disposed on any one of the outer sides or the inner sides of the lower wearing part 100 and the upper wearing part and may be disposed on the outer side and the inner side thereof, respectively, to more precisely measure the balance of force generated from the walker's foot at the time of walking.

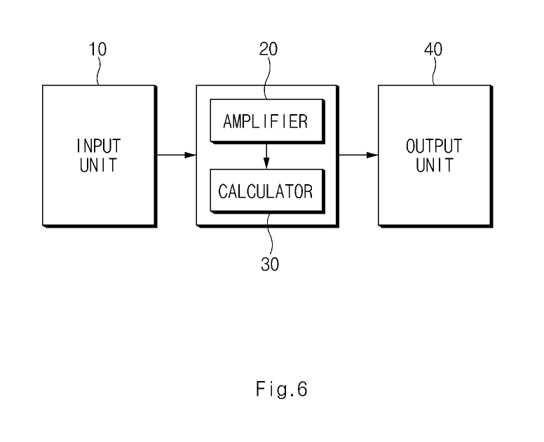

As illustrated in FIG. 6, a walking intension detection system of the walker according to the exemplary embodiment of the present disclosure may include an input unit 10 configured to detect information using the tension sensor 300 in the wearing part into which the walker's foot may be inserted, an amplifier 20 configured to amplify the information into a signal, a calculator 30 configured to detect the walking intention using the signal, and an output unit 40 configured to operate the robot in response to the walking intention. The input device 10, the amplifier 20, the calculator 30, and the output unit 40 may be executed by a controller.

Furthermore, the wearing part and the tension sensor 300 will be described with reference to FIGS. 1 to 5B. The input unit 10 may be configured to detect and measure information regarding the force delivered to the wearing part based on the walking using the tension sensor 300 in the wearing part into which the walker's foot may be inserted. The amplifier 20 may be configured to amplify the information generated from the input unit 10 into a signal. The calculator 30 may be linked with the amplifier 20 to detect the walking intention into the information related signal amplified by the amplifier 20. In particular, the tension sensor 300 may be configured to generate the tension due to the generation of force between the wearing part and the foot in the input unit 10, the amplifier 20 may be configured to amplify the information regarding the tension into the signal, and the calculator 30 may be configured to calculate the force generating point based on the change in the tension sensor 300, thereby detecting the walking intention.

The output unit 40 may be configured to operate the robot in response to the walking intention detected by the calculator 30. In other words, according to the exemplary embodiment of the present disclosure, the walker's foot may be mounted in the wearing part corresponding to the robot's foot when the wearable robot is being worn by a user, the force may be measured by the input unit 10 linked with the tension sensor 300, and the measured data may be transmitted to the amplifier 20 and the calculator 30 to detect the walking intention, to allow the output unit 40 to operate the robot in response to the walking intention.

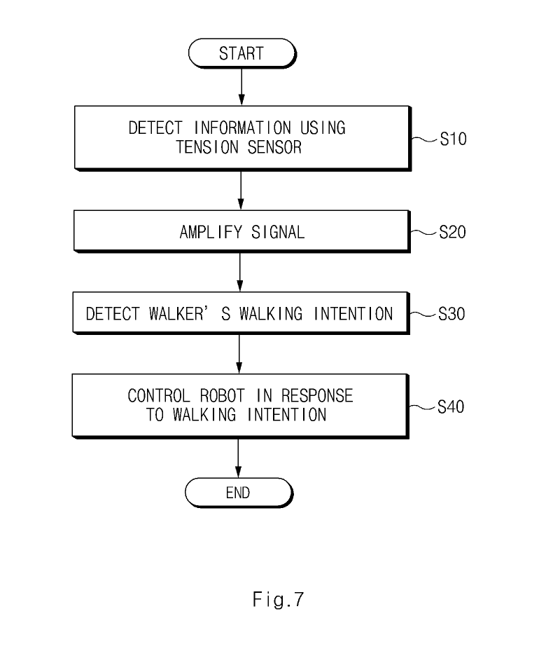

As illustrated in FIG. 7, a walking intension detection method of a walker according to the exemplary embodiment of the present disclosure may include detecting, by a controller, information using a tension sensor 300 in the wearing part into which the walker's foot may be inserted (S10), amplifying, by the controller, the information into the signal (S20), detecting, by the controller, a walking intention using the signal (S30), and operating, by the controller, the robot in response to the walking intention (S40). Meanwhile, the wearing part and the tension sensor 300 will be described with reference to FIGS. 1 to 5B.

In the detection of the information (S10), the information regarding the force delivered to the wearing part in response to the walking motion using the tension sensor 300 in the wearing part into which the walker's foot is inserted may be detected and measured. In the amplification of the information (S20), the information generated in the detection of the information (S10) may be amplified into a signal. In the detection of the walking intention (S30), the walking intention may be detected using the information related signal amplified in the amplifying of the information (S20) which may be operated along with the step S20. In particular, in the detection of the walking intention (S30), the tension sensor 300 may be configured to generate the tension due to the generation of force between the wearing part and the walker's foot in the detection of the information (S10), the information regarding the tension may be amplified into the signal in the amplification of the information (S20), and the force generating point may be calculated based on the change in the tension sensor 300 in the detection of the walking intention (S30), thereby detecting the walking intention.

In the operation of the robot (S40), the robot may be operated in response to the walking intention which may be detected in the detection of the walking intention (S30). That is, according to the exemplary embodiment of the present disclosure, the walker's foot may be mounted in the wearing part corresponding to the robot's foot and thus the tension sensor 300 may be configured to measure the force in the detection of the information (S10), the signal may be amplified in the amplification of the information (S20) and the detection of the walking intention (S30) may be detected based on the amplified signal, and the robot may be operated in response to detecting the walking intention in the operation of the robot (S40).

As described above, the walking intention may be detected when the wearable robot is worn (e.g., mounted to a user) to provide the stability of the walking operation, thereby improving the marketability and convenience and the walking performance of the wearable robot may be increased by the simplified mechanism, thereby creating the higher efficiency at the low costs.

Although the exemplary embodiments of the present disclosure have been disclosed for illustrative purposes, those skilled in the art will appreciate that various modifications, additions and substitutions are possible, without departing from the scope and spirit of the disclosure as disclosed in the accompanying claims. Accordingly, such modifications, additions and substitutions should also be understood to fall within the scope of the present disclosure.

What is claimed is:

1. A walking intention detection device of a walker, comprising:
a lower wearing part into which a walker's foot is inserted;
an upper wearing part disposed over the lower wearing part to cover a top of a foot and a heel of a walker; and
a tension sensor disposed between the lower wearing part and the upper wearing part and configured to measure a force delivered to the lower wearing part and the upper wearing part in response to a walking motion.

2. The walking intention detection device according to claim 1, further comprising:
a controller configured to be linked with the tension sensor to detect a walking intention.

3. The walking intention detection device according to claim 1, wherein the upper wearing part includes a first upper wearing part configured to cover the walker's top of the foot and a second upper wearing part configured to cover the walker's heel and the first upper wearing part and the second upper wearing part are spaced apart from each other at a predetermined interval.

4. The walking intention detection device according to claim 3, wherein the tension sensor includes:
a first load cell disposed between front portions of the first upper wearing part and the lower wearing part; and
a second load cell disposed between rear portions of the second upper wearing part and the lower wearing part.

5. The walking intention detection device according to claim 4, wherein the first load cell includes a first connection band and the second load cell includes a second connection band, and the first connection band of the first load cell connects the first upper wearing part with the lower wearing part and the second connection band of the second load cell connects the second upper wearing part with the lower wearing part.

6. The walking intention detection device according to claim 5, wherein the tension sensor further includes:
a third load cell disposed between the rear portion of the first upper wearing part and the rear portion of the lower wearing part to connect the rear portion of the first upper wearing part with the rear portion of the lower wearing part via a third connection band; and
a fourth load cell disposed between the front portions of the second upper wearing part and the lower wearing part to connect the front portion of the second upper wearing part with the front portion of the lower wearing part via a fourth connection band.

7. The walking intention detection device according to claim 6, wherein the third load cell and the fourth load cell are spaced apart from each other and the third connection band and the fourth connection band intersect each other while being spaced apart from each other.

8. The walking intention detection device according to claim 6, further comprising:
a plurality of rectangular frames disposed between the front portions of the first upper wearing part and the lower wearing part and the rear portions of the second upper wearing part and the lower wearing part,
wherein an inside of the rectangular frame includes the third load cell, the third connection band, the fourth load cell, and the fourth connection band.

9. The walking intention detection device according to claim 4, wherein the tension sensors are disposed on an outer side and an inner side of the lower wearing part and the upper wearing part, respectively, to measure a balance of force generated from the walker's foot during the walking motion.

10. A walking intention detection system of a walker, comprising:
a memory configured to store program instructions; and
a processor configured to execute the program instructions, the program instructions when executed configured to:
sense and measure tension information in a wearing part into which a walker's foot is inserted using a tension sensor;
amplify the tension information into a signal;
detect a walking intention using the amplified signal; and
operate a robot in response to the detected walking intention.

11. A walking intention detection method of a walker, comprising:
sensing and measuring, by a controller, tension information in a wearing part into which a walker's foot is inserted using a tension sensor; and
operating, by the controller, a robot based on the detected tension information.

12. The walking intention detection method according to claim 11, further comprising:
amplifying, by the controller, the tension information into a signal; and
detecting, by the controller, a walking intention using the amplified signal to operate the robot in response to the detected walking intention.

13. A non-transitory computer readable medium containing program instructions executed by a controller, the computer readable medium comprising:
program instructions that sense and measure tension information in a wearing part into which a walker's foot is inserted using a tension sensor;
program instructions that amplify the tension information into a signal;
program instructions that detect a walking intention using the amplified signal; and
program instructions that operate a robot in response to the detected walking intention.
